SEQUENCE LISTING (16 sequences)

SEQ ID NO: 1 - DNA, 20 bp, Artificial
  Primer
aagtagctctgatcaccggc

SEQ ID NO: 2 - DNA, 22 bp, Artificial
  Primer
taactcaatatcctcgtccacc

SEQ ID NO: 3 - DNA, 34 bp, Artificial
  Primer
tagttaacgcgtagttcttggagatggcattcag

SEQ ID NO: 4 - DNA, 34 bp, Artificial
  Primer
tagttaacgcgttaactcaatatcctcgtccacc

SEQ ID NO: 5 - DNA, 23 bp, Artificial
  Primer
gattggtggtattggaactgtcc

SEQ ID NO: 6 - DNA, 19 bp, Artificial
  Primer
gagcttcgtggtgcatctc

SEQ ID NO: 7 - DNA, 34 bp, Artificial
  Primer
tagttatctagacaatcatgaatctcctaggcgg

SEQ ID NO: 8 - DNA, 37 bp, Artificial
  Primer
tagttaggatcctaactcaatatcctcgtccaccatc

SEQ ID NO: 9 - DNA, 34 bp, Artificial
  Primer
tagttagagctccaatcatgaatctcctaggcgg

SEQ ID NO: 10 - DNA, 37 bp, Artificial
  Primer
tagttacccgggtaactcaatatcctcgtccaccatc

SEQ ID NO: 11 - DNA, 27 bp, Artificial
  Primer
atgagtaaacggaatccgaagattctg

SEQ ID NO: 12 - DNA, 22 bp, Artificial
  Primer
ttagcagccaaggctcttcatg

SEQ ID NO: 13 - DNA, 29 bp, Artificial
  Primer
gtgaagaggtttgtgcattttactcattg

SEQ ID NO: 14 - DNA, 26 bp, Artificial
  Primer
tccacccactgcagccaaacaaaaag

SEQ ID NO: 15 - DNA, 41 bp, Artificial
  Primer
tagttaggatccgaggtttgtgcattttactcattgatctg

SEQ ID NO: 16 - DNA, 34 bp, Artificial
  Primer
tagttacccgggccactgcagccaaacaaaaagc

(19) United States
(12) Patent Application Publication
(10) Pub. No.: US 2011/0173720 A1
(43) Pub. Date: Jul. 14, 2011

(54) Method for Modifying Sugar Chain Structure in Plant, and Plant Produced by the Method

(75) Inventors: Kouki Matsuo, Hokkaido (JP); Takeshi Matsumura, Hokkaido (JP)

(73) Assignee: NATIONAL INSTITUTE OF ADVANCED INDUSTRIAL SCIENCE AND TECHNOLOGY, TOKYO (JP)

(21) Appl. No.: 13/060,286

(22) PCT Filed: Aug. 26, 2009
(86) PCT No.: PCT/JP2009/004147

§ 371 Date: Mar. 15, 2011

(30) Foreign Application Priority Data
Aug. 30, 2008 (JP) 2008-222961

(51) Int. Cl. A01H 5/00; C12N 15/82; C12N 5/04; C07K 2/00

(52) U.S. Cl. 800/284; 435/420; 435/410; 800/298; 530/395

(57) ABSTRACT

The present invention provides a method for reducing or inhibiting fucose modification to a N-linked sugar chain in a plant, and a transgenic plant, or the like, in which the fucose modification has been reduced or inhibited. Transcription or translation of a gene coding for an enzyme involved in synthesis of sugar nucleotide GDP-fucose is inhibited by post-transcriptional gene silencing (PTGS), virus-induced gene silencing (VIGS) or transcriptional gene silencing (TGS), and an amount of GDP-fucose in a plant cell is reduced, to reduce or inhibit fucose modification to a sugar chain including glycoprotein sugar chain, glycolipid sugar chain, oligosaccharide, polysaccharide, and the like, thereby enabling production of a glycoprotein, or the like, from which fucose modification, which may become an allergen, has been removed, and enabling clinical application of a medicinal glycoprotein produced with a plant.

### TECHNICAL FIELD

The present invention relates to a method for modifying a sugar chain structure in a plant, which reduces or inhibits fucose modification to a sugar chain including glycoprotein sugar chain, glycolipid sugar chain, oligosaccharide or polysaccharide; more specifically, the invention relates to a method for modifying a plant-type N-linked sugar chain structure, which reduces or inhibits fucose modification to a sugar chain by transforming a plant using a plant transformation vector into which a homologous gene fragment of GMD gene or GER gene has been inserted, which codes for an enzyme involved in the synthesis of GDP-fucose, a species of sugar nucleotide, to a transgenic plant producing a glycoprotein in which fucose modification to sugar chain has been reduced or inhibited, and to a method for synthesizing a glycoprotein in which fucose modification has been reduced or inhibited, using this transformed plant.

### BACKGROUND ART

A sugar chain has various functions owing to the chemical and physical properties thereof. In particular, not only a sugar chain of a glycoprotein is involved in the stability of the protein constituting the glycoprotein, but it is also known that the protein functions for the first time after receiving sugar chain modification. Many glycoproteins receive modification by asparagine-linked sugar chain (hereinafter may be referred to as N-linked sugar chain), in which a sugar chain is linked to an asparagine residue of the protein.

While N-linked sugar chains have a common trimannosyl core structure comprising Man₃GlcNAc₂, in the core sugar chain portion of a plant-type N-linked sugar chain of plant origin, α-1,3-fucose and β-1,2-xylose modifications are present, which are specific to plants and not observed in animal types (FIG. 1).

The possibility has been pointed out that these structures become allergens when administered into the body of an animal (Non-Patent Reference 1), such that removal thereof becomes necessary when a glycoprotein for medicinal use which is administered directly in blood or the like is produced with a transgenic plant, in order to eliminate the allergenicity.

GDP-fucose and UDP-xylose synthesized inside a plant cell are added in the Golgi apparatus to an N-linked sugar chain by the α-1,3-fucose transferase and the β-1,2-xylose gene to form a plant-type sugar chain modification.

So far, for instance, an antibody composition-containing medicine (Patent Reference 1), a genome-modified cell (Patent Reference 2), an antibody composition-producing cell (Patent Reference 3) and an antibody composition preparation method (Patent Reference 4) have been proposed. These patent references describe inhibiting α-1,6-fucose transferase and enzymes involved in GDP-fucose synthesis in order to inhibit α-1,6-fucose addition of animal-type N-linked sugar chain; however, in regard to the GMD gene, merely a lectin-resistant line was selected from animal cells and no gene inhibition regarding plant-type sugar chain by genetic manipulation in plant is performed.

In addition, so far, there are reports regarding inhibition of α-1,3-fucose and β-1,2-xylose modifications to plant-derived N-linked sugar chain. Plants in which α-1,3-fucose transferase and β-1,2-xylose gene are inhibited have been obtained in Arabidopsis (Non-Patent Reference 2), moss (Non-Patent Reference 3) and duckweed (Non-Patent Reference 4) by homologous recombination or mutant selection. Also, plants in which each gene is inhibited have been obtained by the RNAi method, in tobacco (Non-Patent Reference 5).

Meanwhile, with respect to the trimannosyl core structure, when the sugar chain has been extended by an N-acetyl glucosamine on the non-reducing end, there is often an α-1,4-fucose modification on the N-acetyl glucosamine, in plants

In an animal cell or the like, there is often a modification such as β-1,4-galactose on N-acetyl glucosamine; α-1,4-fucose modification in plants competes with β-1,4-galactose modification. Therefore, it is thought that eliminating α-1,4-fucose modification is desirable when producing in a plant a glycoprotein having an animal-type sugar chain modification.

Fucose modification on a sugar chain is by fucose addition effected by various fucose transferases on a sugar chain with GDP-fucose as a fucose donor. In a plant cell, GDP-fucose is synthesized from GDP-mannose by GDP-D-mannose-4,6-dehydratase (GMD) and GDP-4-keto-6-deoxy-D-mannose-3,5-epimerase-4-reductase (GER).

It has been reported that in plants in which the GMD gene has been disrupted, fucose synthesis was no longer possible, constitutive sugars of cell wall polysaccharides were altered, and, in addition, there was no fucose modification on N-linked sugar chain (Non-Patent References 6 and 7). However, the above reports are on mutant selection, and so far, there is no report of inhibition of gene expression carried out in plant by genetic manipulation to transform a plant with the GMD gene or the like as a target.

Since an α-1,3-fucose modification to the reducing end N-acetylglucosamine (GlcNAc) is present in a plant-derived N-linked sugar chain, which is not observed in animal type (it is α-1,6-fucose modification in animal type), methods for inhibiting α-1,6-fucose modification on animal-type sugar chain cannot be applied as-is to a plant-type sugar chain structure. In addition, although there is a report on a CHO cell line in which the GMD gene has been knocked out, it does not necessarily follow that an example from an animal cell can be applied as-is to plants, which genomes are often polyploid.

Various proteins for medicinal use are currently produced by the methods using microorganisms, insect cells, animal cells and the like; however, these methods, from the possibility of contamination by toxins or the like and the aspects of costs, alternative or complementary substance production systems are sought, and as one candidate thereof, development of a substance production system by plant is proceeding world-wide.

In substance production by a plant, in particular, production of glycoprotein for medicinal use, elimination of plant-specific sugar chain modification is sought, owing to the allergenicity thereof. However, the reality is that the findings reported so far are limited to those that inhibit fucose addition to animal-type N-linked sugar chain, use an animal cell, select a mutant, and the like, such that in the technical field, establishment is strongly desired of a technique for inhibiting fucose modification to sugar chain by way of a transformant in which a gene expression has been actually inhibited by applying the virus vector method and transformation with a gene as a target in a plant.

Patent Reference 1: WO 2003/084596 A1

Patent Reference 2: WO 2003/035741 A1

Patent Reference 3: WO 2002/031140 A1

Patent Reference 4: WO 2003/085118 A1

Non-Patent Reference 1: European Journal of Biochemistry 270 (2003) 1327-1337

Non-Patent Reference 2: FEBS Letters, 561 (2004) 132-136

Non-Patent Reference 3: Plant Biotechnology Journal, 5 (2007) 389-401

Non-Patent Reference 4: Nature Biotechnology, 24 (2006) 1591-1597

Non-Patent Reference 5: Plant Biotechnology Journal, 6 (2008) 392-402

Non-Patent Reference 6: Plant Journal, 12 (1997) 335-345

Non-Patent Reference 7: Plant Physiology, 119 (1999) 725-733

In this situation, the present inventors, in view of the aforementioned prior art, performed earnest studies with the objective of taking as the target a gene coding for an enzyme involved in the synthesis of GDP-fucose in plant to carry out inhibition of this gene expression by transforming the plant, and as a result, by taking the GER gene or the GMD gene as the target and by way of the virus vector method and transformation, successfully created a transgenic plant in which fucose modification has been reduced or inhibited by actually reducing or inhibiting this gene expression in the plant, and reached completion of the present invention.

### DISCLOSURE OF THE INVENTION

An object of the present invention is to provide means for reducing/inhibiting fucose modification to a sugar chain, to begin with α-1,3-fucose modification to plant-type N-linked sugar chain, which is a glycoprotein in a plant. In addition, it is an object of the present invention to provide a transgenic plant in which fucose modification to sugar chain has been reduced or inhibited, to begin with α-1,3-fucose modification to plant-type N-linked sugar chain, which is a glycoprotein in a plant. Note that the sugar chain cited here is not limited to a glycoprotein N-linked sugar chain, and includes sugar chains such as glycolipid sugar chain, oligosaccharides and polysaccharides.

The present invention for solving the aforementioned problem is constituted by the technical means below:
(1) A method for modifying a sugar chain structure characterized either by introducing a plant transformation vector having inserted therein a gene fragment inhibiting expression of gene coding for an enzyme involved in synthesis of GDP-fucose, which is a species of sugar nucleotide, into a plant or a plant cell to carry out transformation of the plant, or, infecting a plant or a plant cell with a plant virus vector having inserted therein a gene fragment inhibiting expression of gene coding for an enzyme involved in synthesis of GDP-fucose, thereby inhibiting expression of GDP-fucose synthetase gene to reduce or inhibit fucose modification to a plant-type N-linked sugar chain in the plant,

wherein a DNA sequence of the gene coding for the enzyme, a DNA sequence corresponding to a mutant, an allele, a variant or a homolog of the DNA sequence, or a DNA sequence having at least 50% homology to the gene is used as the gene fragment.
(2) The method according to (1) above, wherein the reduction or inhibition of fucose modification to the plant-type N-linked sugar chain is a reduction or an inhibition of fucose modification to a sugar chain, including a glycoprotein sugar chain, a glycolipid sugar chain, an oligosaccharide or a polysaccharide of a plant.
(3) The method according to (1) or (2) above, wherein the expression of the gene coding for the enzyme involved in the synthesis of GDP-fucose, which is a species of sugar nucleotide, transcription or translation of the gene is blocked by transcriptional gene silencing (TGS), post-transcriptional gene silencing (PTGS) or virus-induced gene silencing (VIGS), thereby blocking intracellular synthesis of GDP-fucose and reducing or inhibiting fucose modification to the sugar chain.
(4) The method according to (1) or (3) above, wherein the gene coding for the enzyme involved in the synthesis of GDP-fucose is a GDP-D-mannose-4,6-dehydratase (GMD) gene or a GDP-keto-6-deoxymannose-3,5-epimerase/4-reductase (GER) gene.
(5) The method according to (1) or (4) above, wherein a plant virus vector is used as the vector for inducing the inhibition of the gene.
(6) The method according to (1) or (4) above, wherein a plant transformation vector is used as the vector for inducing the inhibition of the gene.
(7) A transgenic plant, a plant cell or a plant obtained by the method according to anyone of (1) to (6) above, wherein GDP-fucose synthetase gene expression has been inhibited and fucose modification to a sugar chain has been reduced or inhibited.
(8) The transgenic plant, the plant cell or the plant according to (7) above, wherein the transgenic plant, the plant cell or the plant is a plant cell, a plant or a progeny thereof.
(9) A glycoprotein, a glycopeptide, a glycolipid or a sugar chain obtained from the plant cell, the plant or the progeny thereof defined in (8) above.
(10) A method for synthesizing glycoprotein in which fucose modification to a sugar chain has been reduced or inhibited characterized by using the transgenic plant defined in (7) or (8) above as a host.

Hereafter, the present invention will be described further in detail.

The present invention is a method for modifying a sugar chain structure whereby a plant transformation vector having inserted within a gene fragment inhibiting the gene expression coding for an enzyme involved in the synthesis of GDP-fucose, a species of sugar nucleotide, is introduced into a plant or a plant cell to carry out transformation of the plant, or, a plant or a plant cell is infected with a plant virus vector having inserted within a gene fragment inhibiting the gene expression coding for an enzyme involved in the synthesis of GDP-fucose, thereby inhibiting the expression of GDP-fucose synthetase gene to reduce or inhibit fucose modification to plant-type N-linked sugar chain in a plant, a DNA sequence of a gene coding for the enzyme, a DNA sequence corresponding to a mutant, an allele, a variant or a homolog of the DNA sequence, or, a DNA sequence having at least 50% homology to the gene being used as the gene fragment.

In addition, the point of a transgenic plant, a plant cell or a plant obtained using the method for modifying a sugar chain structure, in which GDP-fucose synthetase gene expression has been inhibited and fucose modification to sugar chain has been reduced or inhibited, in addition, the point of a glycoprotein, a glycopeptide, a glycolipid or a sugar chain obtained from the plant cell, the plant or the progeny thereof, in addition, the point of a method for synthesizing glycoprotein, whereby a glycoprotein in which fucose modification to the sugar chain has been reduced or inhibited is synthesized with the transgenic plant as a host, are characteristics of the present invention.

In the present invention, as a preferred mode of the embodiments, the gene expression coding for the enzyme involved in the synthesis of GDP-fucose, which is a species of sugar nucleotide, transcription or translation of the gene is blocked by transcriptional gene silencing (TGS), post-transcriptional gene silencing (PTGS) or virus-induced gene silencing (VIGS), thereby blocking the intracellular synthesis of GDP-fucose and reducing or inhibiting fucose modification to sugar chain.

In addition, in the present invention, as preferred modes of the embodiments, the reduction or inhibition of fucose modification to the plant-type N-linked sugar chain is a, reduction or an inhibition of fucose modification to a sugar chain, including a glycoprotein sugar chain, a glycolipid sugar chain, an oligosaccharide or a polysaccharide of a plant, in addition, the gene coding for an enzyme involved in the synthesis of GDP-fucose is the GDP-D-mannose-4,6-dehydratase (GMD) gene or the GDP-keto-6-deoxymannose-3,5-epimerase/4-reductase (GER) gene, further, a plant virus vector or a plant transformation vector is used as the vector for inducing inhibition of the gene.

In the present invention, inhibition of an enzyme gene involved in GDP-fucose synthesis can be achieved by taking the mRNA derived from the GMD gene or from the GER gene as the target for destruction, inhibiting the mRNA synthesis per se from the gene, thereby inhibiting the synthesis of the GMD protein or of the GER protein.

When a gene is expressed excessively, plant cells have a mechanism whereby the expression of this gene is inhibited, which functions as a defense mechanism against viruses or the like. In this defense mechanism, the gene expression is inhibited by destroying the over-expressed mRNA. In the present invention, the method of post-transcriptional gene silencing (PTGS) and the method of virus-induced gene silencing (VIGS) can be used.

Next, the method by way of virus-induced gene silencing (VIGS) using the cucumber mosaic virus vector (CMV vector), which is one species of plant virus vector, will be described in detail. In this method, total RNA sample is purified using a leaf sample from tobacco, and a single-strand cDNA is synthesized using the present RNA sample and oligo-dT primers. A partial sequence of the GMD gene is isolated by PCR using the primers NbMD (F) (SEQ ID NO. 1) and NbMD (R) (SEQ ID NO. 2), which are designed based on the known GMD gene sequence.

In order to prepare a gene fragment for GMD gene inhibition, PCR is carried out using the primers CM-NbMD-F (SEQ ID NO. 3) and CM-NbMD-R (SEQ ID NO. 4) having the restriction enzyme MluI site, with the isolated GMD gene as the template. The resulting PCR product is treated with the restriction enzyme MluI, then subjected to agarose gel electrophoresis, the portion of the gene fragment band is excised, and, from the solution containing the gene fragment obtained by centrifugal filtration, the GMD gene fragment to be inserted into the vector is purified.

In order to prepare a CMV vector for GMD gene inhibition, three species of plasmids, pCY1, pCY2(N) and pCY3, for instance, are used as CMV vectors. Among these, pCY2(N) has a multicloning site for the purpose of inserting a gene fragment. pCY2(N) is treated with the restriction enzyme MluI, then, purified and the DNA ends are further dephosphorylated. The dephosphorylated product is purified, then subjected to agarose gel electrophoresis, the portion of the band derived from the pCY2(N) is excised, and, from the solution containing pCY2(N) obtained by centrifugal filtration, the dephosphorylated vector pCY2(N)ΔP is obtained.

With the isolated gene as the template, the gene fragment for GMD gene inhibition is inserted into the dephosphorylated pCY2(N)ΔP. The pCY2(N) plasmid with the gene fragment for GMD gene inhibition inserted (GMD-pCY2(N)) is purified from bacterial body, then, the sequence of the inserted gene fragment is verified, yielding one with the GMD gene fragment inserted into pCY2(N) in the sense orientation (GMD-pCY2(N)S) and one inserted in the antisense orientation (GMD-pCY2(N)A).

In order to prepare a CMV vector for inoculation use to induce VIGS in a plant, it is necessary to infect the plant with a virus. Since the cucumber mosaic virus is an RNA virus, the plant is inoculated with RNA. RNA synthesis for inoculation into N. benthamiana is carried out with GMD-pCY2(N)S, which has the GMD gene fragment inserted, and pCY1, pCY2(N), pCY3 as templates. In order to linearize pCY1, GMD-pCY2(N) S and pCY3, which are circular plasmids, pCY1, pCY2(N) and GMD-pCY2(N)S are subjected to NotI treatment, and pCY3 to EcoRI treatment, to obtain linearized DNAs.

RNA synthesis is carried out by in vitro transcription with the linearized DNAs as the templates. In addition, synthesis of an RNA having a cap structure is carried out by adding Ribo m⁷G Cap Analog (Promega) to the reaction solution. After the end of the reaction, the template DNAs are degraded by DNAse treatment, and the inoculation RNA is purified by phenol treatment and ethanol precipitation. The inoculation RNA is conserved at −80° C. until immediately before inoculation.

In order to inoculate N. benthamiana with the CMV vector for GMD gene inhibition, an RNA solution derived from GMD-pCY2(N) S or GMD-pCY2(N)A is mixed to a mixture of RNAs derived from pCY1 and pCY3 to prepare an RNA solution for inoculation. Similarly, as a negative control, RNA solutions derived from pCY1, pCY2(N) and pCY3 are mixed to prepare an RNA solution for inoculation.

An RNA solution is dripped onto a developed leaf of N. benthamiana at approximately one month after seeding and the entire surface of the leaf is coated with the RNA solution by hand to infect with the CMV vector. A leaf sample is collected three weeks after inoculation and the effect of inhibition of plant-type sugar chain modification is examined. The leaf sample is used to prepare a plant-derived N-linked sugar chain. The sugar chain sample is dissolved in ultrapure water and used in MALDI-TOF-MS analysis.

The evaluation method by MALDI-TOF-MS of the effect of inhibition on plant-type sugar chain modification is as follows. On a target over an MTP AnchorChip™ 600/384 TF plate (Bruker Daltonics), 1 μl of a solution of sugar chain sample and 0.5 μl of an aqueous solution of 2,5-Dihydroxybenzoic Acid (DHB) (5 mg/ml) are mixed and air-dried. Measurements are carried out with Autoflex II TOF/TOF (Bruker Daltonics, reflectron mode, positive ion mode), and the proportion of the peak area for each sugar chain observed (%) is calculated by the formula: Proportion of peak area for sugar chain A (%)=(Peak area of sugar chain A/peak area of all the sugar chains)×100. In the sugar chain samples from plants that are not inoculated and inoculated with the virus vector, the proportions of each sugar chain are compared to evaluate the effect of inhibition on plant-type sugar chain modification.

As an effect of the inhibition of plant-type sugar chain modification by MALDI-TOF-MS, it is observed that α-1,3-fucose modification to N-linked sugar chain decreases from approximately 70% to 20% in plants inoculated with a CMV vector for GMD gene inhibition.

In order to prepare an RNA sample from N. benthamiana, on the third week after virus inoculation, leaf samples are collected from strains inoculated with no virus, strains inoculated with the CMV vector (no insertion of gene for inhibition) and strains inoculated with a CMV vector for GMD gene inhibition, and each leaf sample is used to purify total RNA.

In order to carry out an evaluation by RT-PCR of the expression pattern of the GMD gene, a single-strand cDNA is synthesized using a total RNA sample and oligo-dT primers. PCR is carried out with the present cDNA solution as sample. NbMD (F) (SEQ ID NO. 1) and NbMD (R) (SEQ ID NO. 2) are used as primers for the GMD gene. In addition, with the mRNA derived from the elongation factor-1α (EF-1α) gene as an internal standard, a partial sequence thereof is amplified using the primers EF-1-F (SEQ ID NO. 5) and EF-1-R (SEQ ID NO. 6). In a strain inoculated with a CMV vector for GMD gene inhibition, a remarkable decrease in the amount of mRNA derived from the GMD gene is observed, compared to a non-inoculated strain.

When Real-time PCR is carried out using icycler (Bio-Rad) and iQ SYBR Green Supermix (Bio-Rad) and the amount of mRNA derived from the GMD gene is calculated, a remarkable reduction thereof is observed, at 10% or lower in the strain inoculated with the CMV vector for GMD gene inhibition, compared to the non-inoculated strain.

From this, it is revealed that α-1,3-fucose modification to a glycoprotein N-linked sugar chain derived from plant can be inhibited with VIGS induced by a CMV vector that integrated a GMD gene derived from N. benthamiana. This is thought to be due to the mRNA derived from the GMD gene being destroyed by way of the VIGS induced by infection with a CMV vector for GMD gene inhibition, resulting in the GMD protein in the plant being decreased and the synthesis of GDP-fucose, a fucose donor for the α-1,3-fucose transferase, being inhibited.

Next, a method for inducing PTGS by transforming tobacco plant will be described in detail. In order to prepare a gene fragment for GMD gene inhibition, with the isolated GMD gene as the template, primer pairs having restriction enzyme sites are constructed, and using Nb-iMD-F (Xba) (SEQ ID NO. 7) and Nb-iMD-R (Barn) (SEQ ID NO. 8), and Nb-iMD-F (Sac) (SEQ ID NO. 9) and Nb-iMD-R (Sma) (SEQ ID NO. 10), gene fragments NbiMD (Xba-Bam) and NbiMD (Sma-Sac) of approximately 400 by are obtained by PCR.

NbiMD (Xba-Bam) is treated with the restriction enzymes XbaI and BamHI, then subjected to agarose gel electrophoresis, the portion of the gene fragment band is excised, and, from the solution containing the gene fragment obtained by centrifugal filtration of the gel slice, GMD gene fragment NbiMD (X-B) to be inserted into the vector is obtained. NbiMD (Sma-Sac) is treated with the restriction enzymes SmaI and SacI, and then treated similarly to NbiMD (Xba-Bam) to obtain the GMD gene fragment NbiMD (S-S) to be inserted into the vector.

In order to isolate an intron sequence, a DNA sample is purified from an Arabidopsis thaliana leaf sample. The β-1,2-xylose transferase gene is isolated from the present DNA sample by PCR using primers AtX (F) (SEQ ID NO. 11) and AtX (R) (SEQ ID NO. 12), which are designed based on the known β-1,2-xylose transferase gene sequence (Accession number NM124932).

With the isolated gene as the template, the intron portion is amplified with AtXylt-Int1 (F) (SEQ ID NO. 13) and AtXylt-Intl (R) (SEQ ID NO. 14). With the amplified intron portion DNA as the template, the restriction enzyme site-containing primers AtXTint1-F (Bam) (SEQ ID NO. 15) and AtXTint1-R (Sma) (SEQ ID NO. 16) are used to obtain AtXTint1 (Bam-Sma), which is an intron sequence derived from the A. thaliana β-1,2-xylose transferase gene.

AtXTint1 (Bam-Sma) is treated with the restriction enzymes BamHI and SmaI, then subjected to agarose gel electrophoresis, the portion of the gene fragment band is excised, and, from the solution containing the gene fragment obtained by centrifugal filtration of the gel slice, the intron sequence AtXTint1 (B-S) to be inserted into the vector is obtained.

In order to delete the GUS gene from the plant transformation vector pBE2113, the plant transformation vector pBE2113 is treated with the restriction enzymes SmaI and SacI, then purified, and the DNA ends are further dephosphorylated. The dephosphorylated product is purified, then subjected to agarose gel electrophoresis, the portion of the band derived from pBE2113 is excised, and, from the DNA solution obtained by centrifugal filtration of the gel slice, the gene fragment pBE2113ΔGUS in which the GUS gene has been deleted is obtained.

In order to prepare the plant transformation vector for GMD gene inhibition GMDinv-pBE2113, the GMD gene fragment NbiMD (S-S) is inserted into pBE2113ΔGUS. The vector GMD-pBE2113 with the gene fragment for GMD gene inhibition inserted is purified from the bacterial body and then the sequence of the inserted gene fragment is verified.

GMD-pBE2113 is treated with the restriction enzymes BamHI and SmaI, then purified, and the DNA ends are further dephosphorylated. The dephosphorylated product is purified, then subjected to agarose gel electrophoresis, the portion of the band derived from GMD-pBE2113 is excised, and, from the DNA solution obtained by centrifugal filtration of the gel slice, GMD-pBE2113ΔP is purified.

The intron sequence AtXTint1 (B-S) is inserted into GMD-pBE2113ΔP. The vector INT-GMD-pBE2113 with the intron sequence AtXTiht1 (B-S) inserted is purified from the bacterial body and then the sequence of the inserted gene fragment is verified.

INT-GMD-pBE2113 is treated with the restriction enzymes XbaI and BamHI, then purified, and the DNA ends are further dephosphorylated. The dephosphorylated product is purified, then subjected to agarose gel electrophoresis, the portion of the band derived from INT-GMD-pBE2113 is excised, and, from the DNA solution obtained by centrifugal filtration of the gel slice, INT-GMD-pBE2113ΔP is purified.

The GMD gene fragment NbiMD (X-B) is inserted into INT-GMD-pBE2113ΔP. The vector GMDinv-pBE2113 with the GMD gene fragment NbiMD (X-B) inserted is purified from the bacterial body, and a sequence analysis of the inserted gene fragment is performed to verify that the GMD gene fragments are flanking the intron sequence and forming an inverted repeat sequence.

Agrobacterium tumefaciens LBA4404 strain is used to carry out the transformation of the tobacco Nicotiana benthamiana by way of the leaf disc method. From a tobacco leaf, a leaf disc is cut out with a cork borer, which is soaked in a bacterial solution of Agrobacterium tumefacience LBA4404 strain having GMDinv-pBE2113, and co-cultured on MS agar medium to let Agrobacterium infect the tobacco leaf.

Agrobacterium bacterial body is eliminated by washing the leaf disc with an MS liquid medium containing kanamycin and carbenicillin. The leaf disc is cultured over a redifferentiation MS agar medium added with these antibiotics to obtain a transformed tobacco shoot. The shoot is cultured in a rooting medium containing kanamycin and carbenicillin, and grown.

In order to evaluate by MALDI-TOF-MS the effect of the inhibition of plant-type sugar chain modification, N-linked sugar chain is purified from the obtained transformed plant, and a sugar chain structure analysis by MALDI-TOF-MS is carried out to evaluate the extent of fucose modification inhibition. It is observed that α-1,3-fucose modification to N-linked sugar chain decreases from 70% to 5% in the Mdi-11 strain.

In order to evaluate by RT-PCR the expression pattern of the GMD gene, a single-strand cDNA is synthesized using a total RNA sample and oligo-dT primers. PCR is carried out with the present cDNA solution as sample. NbMD (F) (SEQ ID NO. 1) and NbMD (R) (SEQ ID NO. 2) are used as primers for the GMD gene. In addition, with the mRNA derived from the elongation factor-1α gene as an internal standard, a partial sequence thereof is amplified using the primers EF-1-F (SEQ ID NO. 5) and EF-1-R (SEQ ID NO. 6). In transformed tobacco, a remarkable decrease in the amount of mRNA derived from the GMD gene is observed, compared to untransformed tobacco.

In the present invention, it is possible to utilize the PTGS method and the VIGS method as methods for inhibiting gene expression by utilizing the mechanism of gene expression inhibition in a plant. Inhibition of a specific gene becomes possible by excessively expressing in the plant cell a gene sequence that is homologous to the gene which inhibition is desired. It suffices for the methods that induce PTGS and VIGS to be methods that are able to express in a plant cell a gene sequence that is homologous to the gene which inhibition is desired, and methods such as transformation and utilizing a virus vector can be utilized.

In the present invention, it is possible to use as vectors for inducing inhibition of the above genes, plant virus vectors, for instance, CMV vector (Plant Biotechnol. J., 2007, November; 5(6):778-90), tobacco rattle virus (J. Biol. Chem., 2006, May 12; 281 (19):13708-16, The Plant Journal (2001) 25 (2), 237-245), Potato virus vector (Plant Physiology, April 2004, Vol. 134, pp. 1308), in addition, plant transformation vectors, for example, pBE2113 (Genetics, Vol. 160, 343-352, January 2002.), pKANNIBAL (BMC Biotechnol., 2008, Apr. 3; 8:36), pBE2113 and pHELLSGATE8 (Plant Physiology, November 2005, Vol. 139, pp. 1175), pBI121 (Plant Physiology, July 2001, Vol 126 pp. 965), various transformation vectors (Journal of Integrative Plant Biology 2007, 49(4) :556-567), and the like. In the present invention, it is possible to use suitable plasmid vectors and virus vectors as plant transformation vectors and plant virus vectors.

In plant, mainly, gene inactivation of the transcription inhibition type [transcriptional gene silencing (TGS)], in which the promoter region of the gene becomes methylated, is also carried out. In TGS, induction is possible by excessively expressing inside a plant cell, for example, a DNA sequence containing the promoter region of the gene which inhibition is desired, as-is, or as to form an inverted repeat sequence (EMBO Journal 19 (2000) 5194-5201 or the like).

In the present invention, it is possible to use as gene sequences for inhibiting the GMD gene or the GER gene, a gene sequence coding for the GMD protein or the GER protein per se, for instance, the GMD gene (partial) sequence from N. benthamiana (NCBI, Accession Number: CN747648, CN747739, U81805), the GMD gene sequence from Arabidopsi thaliana (NCBI, Accession Number: U81805, NM_{—}126026, NM_{—}114976), and the like. In addition, it is also possible to use a DNA coding for a protein that is functionally similar to the GMD protein or the GER protein, for instance, a mutant, an allele, a variant, a homolog or the like, in addition, a DNA sequence having 50% or greater homology with the DNA sequence coding for the GMD gene or the GER gene, furthermore, a DNA sequence that is capable of inhibiting transcription of the gene by being methylated, and the like.

According to the present invention, effects such as the following are produced:

(1) By reducing or inhibiting fucose modification to glycoprotein N-linked sugar chain in plant, a method for modifying a plant-type sugar chain structure can be provided.

(2) A transgenic plant can be prepared and provided, in which fucose modification to glycoprotein N-linked sugar chain is reduced or inhibited.

(3) By utilizing the transgenic plant as a host, synthesis of a glycoprotein in which the sugar chain structure of the glycoprotein is modified becomes possible.

(4) By utilizing the transgenic plant of the present invention, production of a glycoprotein becomes possible, in which plant-specific fucose modification having the possibility of becoming an allergen when administered in an organism is reduced or inhibited.

(5) With the transgenic plant, production and provision of a medicinal glycoprotein in which allergenicity is decreased become possible.

### BRIEF DESCRIPTION OF THE DRAWINGS

FIG. 1 shows the differences in glycoprotein N-linked sugar chain modifications on a plant-type sugar chain and an animal-type sugar chain, and on the plant-type, α-1,3-fucose and β-1,2-xylose modifications are present.

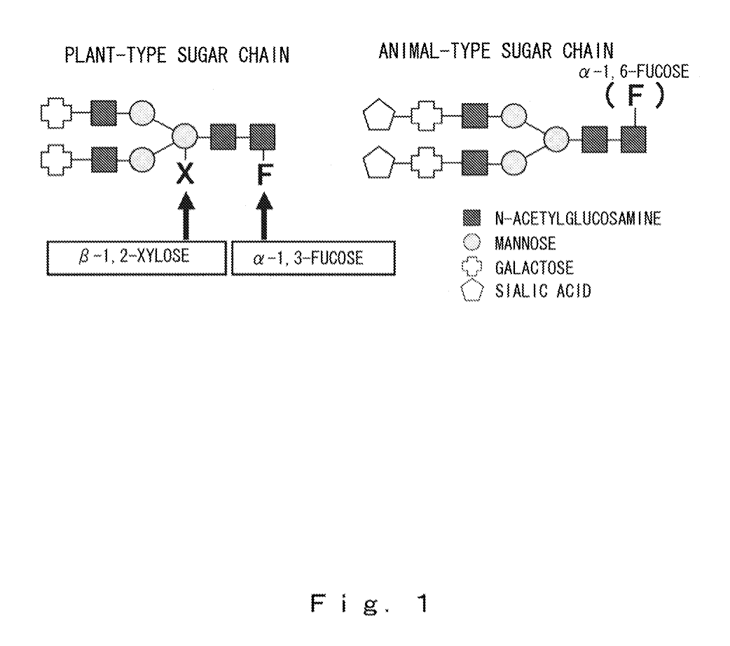

FIG. 2 shows the cucumber mosaic virus (CMV) vectors pCY1, pCY2(N) and pCY3 used for inhibiting plant-type sugar chain modification.

FIG. 3 shows effect of inhibiting fucose modification to N-linked sugar chain in the plant by the CMV vector for GMD gene inhibition.

FIG. 4 shows the analysis results by RT-PCR of the GMD gene expression pattern in each virus vector-inoculated plant.

FIG. 5 shows the abundance ratio of mRNA derived from the GMD gene by Real-time PCR.

FIG. 6 shows the plant transformation vector GMDinv-pBE2113 designed for GMD gene inhibition.

FIG. 7 shows the results after purifying N-linked sugar chain from a transformant, carrying out sugar chain structure analysis by MALDI-TOF-MS and evaluating the extent of fucose modification inhibition.

FIG. 8 shows the analysis results by RT-PCR of GMD gene expression pattern in a GMD gene inhibited recombinant plant.

### BEST MODE FOR CARRYING OUT THE INVENTION

Next, examples will be shown to describe the present invention concretely; however, the following examples indicate preferred examples of the present invention and do not limit the scope of the present invention in terms of plant species, methods for inducing PTGS, VIGS and TGS, or the like.

### EXAMPLE 1

In the present example, fucose modification to sugar chain was carried out by virus-induced gene silencing (VIGS) using the cucumber mosaic virus vector (CMV vector), which is one species of plant virus vector.

(1) Isolation of GMD Gene from Tobacco Nicotiana benthamiana

Nicotiana benthamiana leaf sample, which is a species of tobacco, was ground in liquid nitrogen to purify total RNA sample thereof with the RNeasy Plant Mini Kit (Qiagen). A single-strand cDNA was synthesized with the Ready-To-Go RT-PCR Beads (GE Health Care) using the present RNA sample and oligo-dT primers.

A partial sequence of the GMD gene was isolated by PCR using the primers NbMD (F) (SEQ ID NO. 1: 5′-AAGTAGCTCTGATCACCGGC) and NbMD (R) (SEQ ID NO. 2: 5′-TAACTCAATATCCTCGTCCACC), which were designed based on the known GMD gene sequence (Accession numbers CN747648 and CN747739). For the PCR, after treatment threreof at 94° C. for 3 minutes, the cycle 98° C. (30 seconds)→56° C. (15 seconds)→68° C. (2 minutes) was repeated 35 times by using KOD-plus-Ver. 2 (TOYOBO).

(2) Preparation of Gene Fragment for GMD Gene Inhibition

A gene fragment of approximately 200 by was obtained by PCR with KOD-plus-Ver. 2 (TOYOBO) using the primers CM-NbMD-F (SEQ ID NO. 3: 5′-TAGTTAACGCGTAGTTCTTGGAGATGGCATTCAG) and CM-NbMD-R (SEQ ID NO. 4: 5′-TAGTTAACGCGTTAACTCAATATCCTCGTCCACC) having the restriction enzyme MluI site With the isolated GMD gene as template. For the PCR, after treatment thereof at 94° C. for 3 minutes, the cycle 98° C. (30 seconds)→56° C. (15 seconds)→68° C. (1 minute) was repeated 35 times.

The resulting PCR product was treated with the restriction enzyme MluI, then subjected to agarose gel electrophoresis, and the portion of the gene fragment band was excised. A solution containing the gene fragment was obtained by filtering the gel slice by centrifugation (5,000×g, 5 minutes) with an Ultrafree-MC Filter Unit (Millipore). The GMD gene fragment to be inserted into the vector was purified from the present solution.

(3) Preparation of CMV Vector for GMD Gene Inhibition

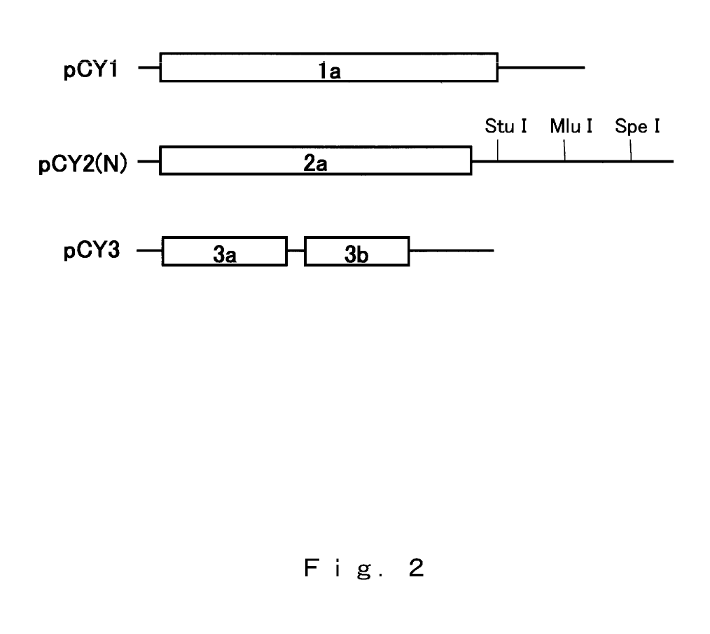

The CMV vector used in the present example is constituted by three species of plasmids, pCY1, pCY2(N) and pCY3, and among these, pCY2(N) has a multicloning site for the purpose of inserting a gene fragment (FIG. 2 and Japanese Patent Application Laid-open No. 2005-013164).

pCY2(N) was treated with the restriction enzyme MluI, then, purified and the DNA ends were further dephosphorylated with E. coli Alkaline Phosphatase (TOYOBO). The dephosphorylated product was purified, then subjected to agarose gel electrophoresis, the portion of the band derived from the pCY2(N) was excised, and a solution containing pCY2(N) was obtained by filtering the gel slice by centrifugation (5,000×g, 5 minutes) with an Ultrafree-MC Filter Unit (Millipore). the dephosphorylated vector pCY2(N)ΔP was obtained from the present solution.

With the isolated gene as a template, insertion of the gene fragment for GMD gene inhibition into the dephosphorylated pCY2(N)ΔP was carried out with the DNA Ligation Kit Ver. 2.1 (Takara Bio). The ligation reaction solution was used as-is for transformation of Escherichia coil DH5α competent cell (Takara Bio).

The pCY2(N) plasmid with the gene fragment for GMD gene inhibition inserted (GMD-pCY2(N)) was purified from the bacterial body with the Wizard Plus Minipreps DNA Purification System (Promega), then, the sequence of the inserted gene fragment was verified, yielding one with the GMD gene fragment inserted into pCY2(N) in the sense orientation (GMD-pCY2(N)S) and one inserted in the antisense orientation (GMD-pCY2(N)A).

(4) Preparation of CMV Vector for Inoculation Use

In order to induce VIGS in plant, it is necessary to infect the plant with a virus. Since the cucumber mosaic virus is an RNA virus, the plant is to be inoculated with RNA.

RNA synthesis for inoculation into N. benthamiana was carried out with GMD-pCY2(N)S, which had the GMD gene fragment inserted, and pCY1, pCY2(N), pCY3 as templates. First, in order to linearize pCY1, GMD-pCY2(N)SandpCY3, which are circular plasmids, pCY1, pCY2(N) and GMD-pCY2(N)S were subjected to NotI treatment, and pCY3 to EcoRI treatment, to obtain linearized DNAs.

With the linearized DNAs as the templates, RNA synthesis was carried out by in vitro transcription with the RiboMAX Large Scale RNA Production System-T7 (Promega). In addition, synthesis of an RNA having a cap structure is carried out by adding Ribo m⁷G Cap Analog (Promega) to the reaction solution.

After the end of the reaction, the template DNAs were degraded by DNAse treatment, and the RNA for inoculation was purified by phenol treatment and ethanol precipitation. The RNA for inoculation was dissolved in RNase-free water and conserved at −80° C. until immediately before inoculation. In addition, in order to inhibit RNA degradation as much as possible, synthesis of the RNA for inoculation was carried out on the day of inoculation.

(5) Inoculation of N. benthamiana with the CMV Vector for GMD Gene Inhibition

Inoculation of N. benthamiana with RNA (infection with the CMV vector) was carried out as described in the following. An RNA solution derived from GMD-pCY2(N)S or GMD-pCY2(N)A was mixed to a mixture of RNAs derived from pCY1 and pCY3 to prepare an RNA solution for inoculation. Similarly, as a negative control, RNA solutions derived from pCY1, pCY2(N) and pCY3 were mixed to prepare an RNA solution for inoculation.

Two developed leaves of N. benthamiana at approximately one month after seeding were coated with small amounts of Carborundum (Nacalai Tesque), an RNA solution was dripped thereon with a Pipetman and the entire surface of the leaf was coated with the RNA solution by a hand wearing a rubber glove to infect with the CMV vector. A leaf sample was collected three weeks after inoculation and the effect of inhibition of plant-type sugar chain modification was examined.

(6) Preparation of Plant-Derived N-Linked Sugar Chain

The preparation of plant-derived N-linked sugar chain was carried as described in the following. Using a mortar and a pestle, 50 mg of leaf sample was ground and the grounds were suspended in 2 ml of 100 mM Tris-HCl buffer (pH 7.5). The suspension was centrifuged at 3,000 rpm for 10 minutes, and then the supernatant was filtered with a Minisart syringe filter (0.2 μm, Sartorius).

After dripping and mixing 600 μl of 50% trichloroacetic acid to the filtrate, the solution was let to stand still overnight at 4° C. The sample solution was centrifuged at 3,000 rpm for 10 minutes to precipitate proteins. The precipitated proteins were washed three times with an aqueous solution of 90% acetone and then dried under reduced pressure in a 1.5 ml Eppendorf tube. After 50 μl of deionized water was added, the dried proteins were treated at 100° C. for 10 minutes.

To the present solution, 50 μl of 0.02 M HCl was added to re-suspend the proteins. To the suspension, 20 μl of a pepsin solution (5 mg/ml (0.01 M HCl)) was added and the solution was let to stand still at 37° C. for two hours. Thereafter, further 20 μl of the pepsin solution was added, and the solution was let to stand still overnight at 37° C. After neutralizing the pepsin treatment solution with 1 M ammonia water, pepsin was inactivated by 10-minute treatment at 100° C.

The sample solution was centrifuged at 16,000 rpm for 10 minutes, the supernatant was transferred to a new 1.5 ml Eppendorf tube and dried under reduced pressure. The dry solid was suspended in 47.5 μl of 0.5 M citric acid buffer (pH 5.0), then, glycopeptidase A (0.05 mU/2.5 μl, Seikagaku Corporation) dissolved in 0.1 M citric acid buffer (pH 5.0) was added, and the solution was let to stand still overnight at 37° C.

To the glycopeptidase A treatment solution, 50 μl of 1M Tris-HCl buffer (pH 8.0) was added to treat the solution at 100° C. for 10 minutes. The sample solution was centrifuged at 16,000 rpm for 10 minutes, the supernatant was purified and desalted by sequentially treating with Dowex 50 (H⁺) column, Dowex 1 (CO₃²⁻) column and Carbograph column, and then dried under reduced pressure to obtain a sugar chain sample. The sugar chain sample was dissolved in 10 μl of ultra-pure water and used for MALDI-TOF-MS analysis.

(7) Evaluation Method by MALDI-TOF-MS of the Effect of Inhibition on Plant-Type Sugar Chain Modification

On a target over an MTP AnchorChip™ 600/384 TF plate (Bruker Daltonics), 1 μl of a solution of sugar chain sample and 0.5 μl of an aqueous solution of 2,5-Dihydroxybenzooic Acid (DHB) (5 mg/ml) were mixed and air-dried. Measurements were carried out with Autoflex II TOF/TOF (Bruker Daltonics, reflectron mode, positive ion mode), and the proportion (%) of the peak area for each sugar chain observed was calculated with the following formula:

Proportion of peak area for sugar chain A (%)=(Peak area of sugar chain A/peak area of all the sugar chains)×100

In the sugar chain samples from plants that were not inoculated and inoculated with the virus vector, the proportions of each sugar chain were compared to evaluate the effect of inhibition on plant-type sugar chain modification.

(8) Evaluation by MALDI-TOF-MS of the Effect of Inhibition on Plant-Type Sugar Chain Modification

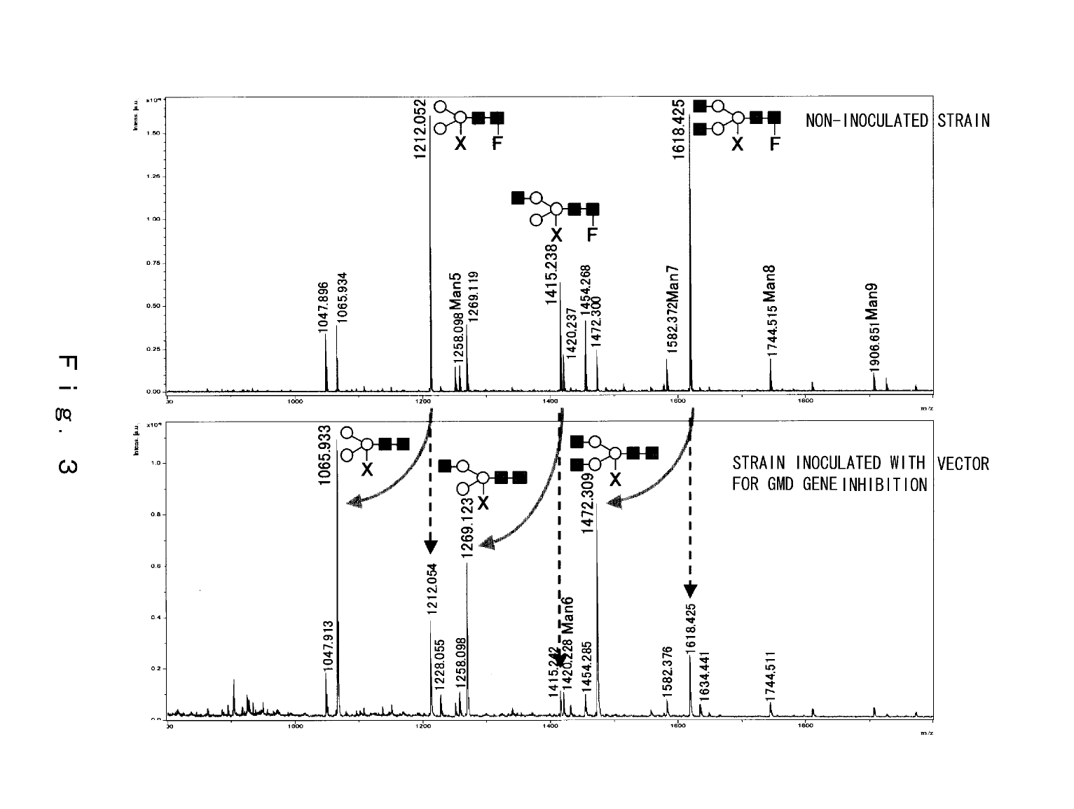

It was observed that α-1,3-fucose modification to N-linked sugar chain decreases from approximately 70% to 20% in plants inoculated with a CMV vector for GMD gene inhibition (FIG. 3 and Table 1)

TABLE 1

| | Abundance ratio of | |
|---|---|---|
| | {illegible} sugar chain (%) | |
| | | Strain inoculated |
| | | with GMD gene |
| Proposed structure of sugar chain | WT | inhibition vector |
| Man3XylGlcNAc | 0.25 | 0.39 |
| Man2XylGlcNAc2 | 0.00 | 2.78 |
| Man3GlcNAc2 | 0.24 | 0.85 |
| Man2XylGucGlcNAc2 | 4.11 | 2.30 |
| Man2XylFucGlcNAc2 | 1.08 | 0.00 |
| Man3XylGlcNAc2 | 5.26 | 23.34 |
| Man3FucGlcNAc2 | 0.00 | 0.25 |
| GlcNAcMan2XylGlcNAc2 | 0.41 | 0.00 |
| GlcNAcMan3GlcNAc2 | 0.20 | 0.45 |
| Man3XylFucGlcNAc2 | 21.35 | 8.17 |
| Man4XylGlcNAc2 | 0.42 | 1.81 |
| GlcNAcMan2XylFucGlcNAc2 | 1.96 | 0.76 |
| Man5 | 2.00 | 2.09 |
| GlcNAcMan3XylGlcNAc2 | 5.24 | 13.41 |
| GlcNAc2Man3GlcNAc2 | 0.34 | 0.72 |
| GlcNAcMan3XylFucGlcNAc2 | 9.77 | 2.58 |
| Man6 | 2.83 | 2.30 |
| HexHexNAcMan3XylGlcNAc2 | 0.00 | 1.09 |
| GlcNAc2Man2XylFucGlcNAc2 | 5.82 | 1.82 |
| GlcNAc2Man3XylGlcNAc2 | 3.46 | 22.87 |
| HexGlcNAcMan3XylFucGlcNAc2 | 0.48 | 0.00 |
| Man7 | 2.60 | 1.06 |
| GlcNAc2Man3XylFucGlcNAc2 | 25.91 | 5.12 |
| HexHexNAc2Man3XylGlcNAc2 | 0.17 | 1.37 |
| HexGlcNAc2Man3FucGlcNAc2 | 0.36 | 0.32 |
| Man8 | 2.64 | 1.80 |
| Hex2GlcNAc2Man3FucGlcNAc2 | 0.90 | 0.65 |
| Man9 | 1.70 | 1.34 |
| FucHexGlcNAc2Man3XylFucGlcNAc2 | 1.04 | 0.14 |
| Hex3GlcNAc2Man3FucGlcNAc2 | 0.47 | 0.21 |
| Total | | |
| Fuc modification present | 72.25 | 22.32 |
| No Fuc | 15.99 | 69.08 |
| High-mannose | 11.77 | 8.59 |

(9) Preparation of RNA Sample from N. benthamiana

On the third week after virus inoculation, leaf samples were collected from strains inoculated with no virus, strains inoculated with the CMV vector (no insertion of gene for inhibition) and strains inoculated with a CMV vector for GMD gene inhibition, and each leaf sample was ground in liquid nitrogen, and total RNA sample was purified with the RNeasy Plant Mini Kit (Qiagen).

(10) Evaluation by RT-PCR of the Expression Pattern of the GMD Gene

A single-strand cDNA was synthesized with the Ready-To-Go RT-PCR Beads (GE Health Care) using 5 μg of total RNA sample and oligo-dT primers. PCR using GoTaq Green Master Mix (Promega) and T-personal Thermal Cycler (Biometra) was carried out with 1 μl of the present cDNA solution as sample. For the PCR, after treatment at 94° C. for 3 minutes, the cycle 94° C. (30 seconds)→56° C. (30 seconds)→72° C. (1 minute 30 seconds) was repeated 35 times.

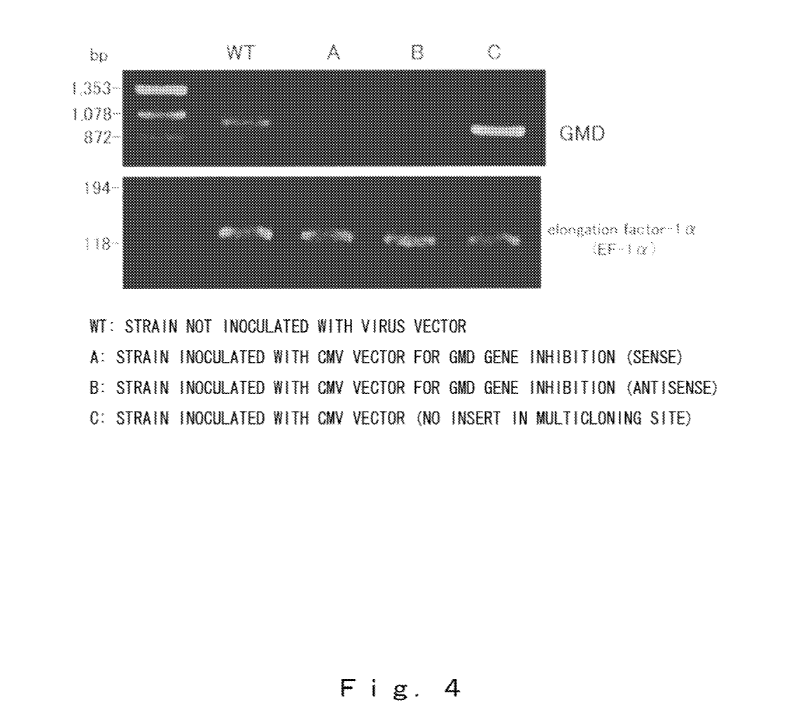

As GMD gene primers, NbMD (F) (SEQ ID NO. 1: 5′-AAGTAGCTCTGATCACCGGC) and NbMD (R) (SEQ ID NO. 2: 5′-TAACTCAATATCCTCGTCCACC) were used. In addition, with the mRNA derived from the elongation factor-1α (EF-1α) gene as an internal standard, a partial sequence thereof was amplified using the primers EF-1-F (SEQ ID NO. 5: 5′-GATTGGTGGTATTGGAACTGTCC) and EF-1-R (SEQ ID NO. 6: 5′-GAGCTTCGTGGTGCATCTC). As a result, in a strain inoculated with a CMV vector for GMD gene inhibition, a remarkable decrease in the amount of mRNA derived from the GMD gene was observed, compared to a non-inoculated strain (FIG. 4).

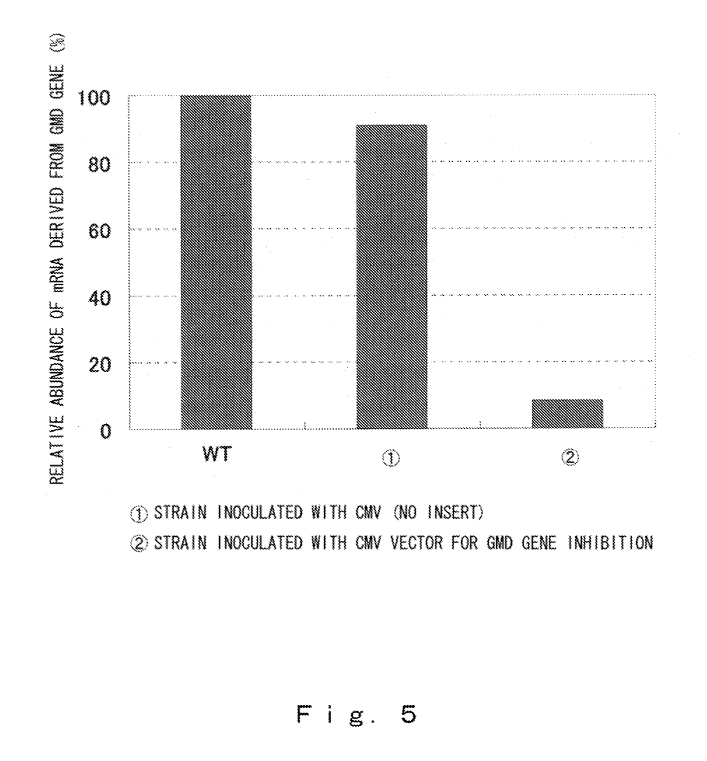

Real-time PCR was carried out using icycler (Bio-Rad) and iQ SYBR Green Supermix (Bio-Rad). The amount of mRNA derived from the GMD gene was calculated, and as a result, a remarkable reduction thereof was observed, at 10% or lower in the strain inoculated with the CMV vector for GMD gene inhibition, compared to the non-inoculated strain (FIG. 5).

From the above results, it has become clear that α-1,3-fucose modification to a glycoprotein N-linked sugar chain derived from plant can be inhibited with VIGS induced by a CMV vector that integrated a GMD gene derived from N. benthamiana. This is inferred to be due to the mRNA derived from the GMD gene being destroyed by way of the VIGS induced by infection with a CMV vector for GMD gene inhibition, resulting in the GMD protein in the plant being decreased and the synthesis of GDP-fucose, a fucose donor for the α-1,3-fucose transferase, being inhibited.

### EXAMPLE 2

In the present example, inhibition of fucose modification to sugar chain was carried out by transforming a tobacco plant to induce PTGS.

(1) Preparation of Gene Fragment for GMD Gene Inhibition

With the isolated GMD gene as the template, Nb-iMD-F (Xba) (SEQ ID NO. 7: TAGTTATCTAGACAATCATGAATCTCCTAGGCGG) and Nb-iMD-R (Bam) (SEQ ID NO. 8: TAGTTAGGATCCTAACTCAATATCCTCGTCCACCATC), Nb-iMD-F (Sac) (SEQ ID NO. 9: TAGTTAGAGCTCCAATCATGAATCTCCTAGGCGG) and Nb-iMD-R (Sma) (SEQ ID NO. 10: TAGTTACCCGGGTAACTCAATATCCTCGTCCACCATC) were used as primer pairs having restriction enzyme sites, and gene fragments NbiMD (Xba-Bam) and NbiMD (Sma-Sac) of approximately 400 by were obtained by PCR using KOD-plus-Ver. 2 (TOYOBO). For the PCR, after treatment thereof at 94° C. for 3 minutes, the cycle 98° C. (30 seconds)→56° C. (15 seconds)→68° C. (1 minute 30 seconds) was repeated 35 times.

NbiMD (Xba-Bam) was treated with restriction enzymes XbaI and BamHI, then subjected to agarose gel electrophoresis, and the portion of the gene fragment band was excised. A solution containing the gene fragment was obtained by filtering the gel slice by centrifugation (5,000×g, 5 minutes) with an Ultrafree-MC Filter Unit (Millipore). The GMD gene fragment NbiMD (X-B) to be inserted into the vector was purified from the present solution.

NbiMD (Sma-Sac) was treated with the restriction enzymes SmaI and SacI, and then treated similarly to NbiMD (Xba-Bam) to obtain the GMD gene fragment NbiMD (S-S) to be inserted into the vector.

(2) Isolation of Intron Sequence

A DNA sample was purified from an Arabidopsis thaliana leaf sample with the DNeasy Plant Mini Kit (Quiagen). The β-1,2-xylose transferase gene is isolated from the present DNA sample by PCR using primers AtX (F) (SEQ ID NO. 11: 5′-ATGAGTAAACGGAATCCGAAGATTCTG) and AtX (R) (SEQ ID NO. 12: 5′-TTAGCAGCCAAGGCTCTTCATG), which were designed based on the known β-1,2-xylose transferase gene sequence (Accession number NM124932). For the PCR, after treatment thereof at 94° C. for 3 minutes, the cycle 98° C. (30 seconds)→56° C. (15 seconds)→68° C. (2 minutes) was repeated 35 times by using KOD-plus-Ver. 2 (TOYOBO).

With the isolated gene as the template, the intron portion was amplified with AtXylt-Int1 (F) (SEQ ID NO. 13: 5′-GT GAAGAGGTTT GTGCATTTTA CTCATTG) and AtXylt-Int1 (R) (SEQ ID NO. 14: 5′-TCCACCCACTGCAGCCAAACAAAAAG).

With the amplified intron portion DNA as the template, the restriction enzyme site-containing primers AtXTint1-F (Bam) (SEQ ID NO. 15: 5′-TAGTTA GGATCC GAGGTTT GTGCATTTTA CTCATTGATC TG) and AtXTint1-R (Sma) (SEQ ID NO. 16: 5′-TAGTTACCCGGG CCACTGCAGCCAAACAAAAAGC) were used to obtain AtXTint1 (Bam-Sma), which is an intron sequence derived from the A. thaliana β-1,2-xylose transferase gene.

AtXTint1 (Bam-Sma) is treated with the restriction enzymes BamHI and SmaI, then subjected to agarose gel electrophoresis, the portion of the gene fragment band was excised. A solution containing the gene fragment was obtained by filtering the gel slice by centrifugation (5,000×g, 5 minutes) with an Ultrafree-MC Filter Unit (Millipore). The intron sequence AtXTint1 (B-S) to be inserted into the vector was purified from the present solution.

(3) Deletion of the GUS Gene from the Plant Transformation Vector pBE2113

The plant transformation vector pBE2113 was treated with the restriction enzymes SmaI and SacI, then purified, and the DNA ends were further dephosphorylated with E. coli Alkaline Phosphatase (TOYOBO).

The dephosphorylated product was purified, then subjected to agarose gel electrophoresis, the portion of the band derived from pBE2113 was excised, and a DNA solution was obtained by filtering the gel slice by centrifugation (5,000×g, 5 minutes) with an Ultrafree-MC Filter Unit (Millipore). The gene fragment pBE2113ΔGUS in which the GUS gene was deleted was obtained from the present solution.

(4) Preparation of the Plant Transformation Vector for GMD Gene Inhibition GMDinv-pBE2113

The GMD gene fragment NbiMD (S-S) was inserted into pBE2113ΔGUS with the DNA Ligation Kit Ver. 2.1 (Takara Bio). The ligation reaction solution was used as-is for transformation of Escherichia coli DH5a competent cell (Takara Bio). The vector GMD-pBE2113 having the gene fragment for GMD gene inhibition inserted was purified from the bacterial body with the Wizard Plus Minipreps DNA Purification System (Promega), and then, the sequence of the inserted gene fragment was verified.

GMD-pBE2113 was treated with the restriction enzymes BamHI and SmaI, then purified, and the DNA ends were further dephosphorylated with E. coli Alkaline Phosphatase (TOYOBO). The dephosphorylated product was purified, then subjected to agarose gel electrophoresis, the portion of the band derived from GMD-pBE2113 was excised, and a DNA solution was obtained by filtering the gel slice by centrifugation (5,000×g, 5 minutes) with an Ultrafree-MC Filter Unit (Millipore). The GMD-pBE2113ΔP was purified from the present solution.

The intron sequence AtXTint1 (B-S) was inserted into GMD-pBE2113ΔP with the DNA Ligation Kit Ver. 2.1 (Takara Bio). The ligation reaction solution was used as-is for transformation of Escherichia coli DH5α competent cell (Takara Bio). The vector INT-GMD-pBE2113 having the intron sequence AtXTint1 (B-S) inserted was purified from the bacterial body with the Wizard Plus Minipreps DNA Purification System (Promega), and then, the sequence of the inserted gene fragment was verified.

INT-GMD-pBE2113 was treated with the restriction enzymes XbaI and BamHI, then purified, and the DNA ends were further dephosphorylated with E. coli Alkaline Phosphatase (TOYOBO). The dephosphorylated product was purified, then subjected to agarose gel electrophoresis, the portion of the band derived from INT-GMD-pBE2113 was excised, and a DNA solution was obtained by filtering the gel slice by centrifugation (5,000×g, 5 minutes) with an Ultrafree-MC Filter Unit (Millipore). INT-GMD-pBE2113ΔP was purified from the present solution.

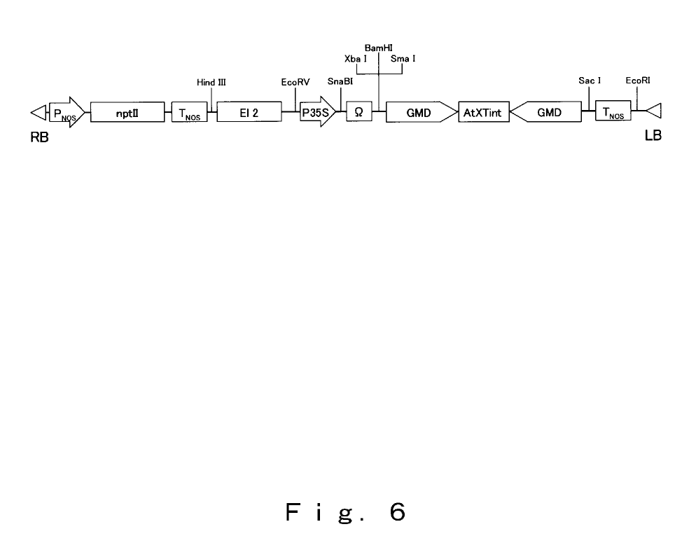

The GMD gene fragment NbiMD (X-B) was inserted into INT-GMD-pBE2113ΔP with the DNA Ligation Kit Ver. 2.1 (Takara Bio). The ligation reaction solution was used as-is for transformation of Escherichia coli DH5α competent cell (Takara Bio). The vector GMDinv-pBE2113 (FIG. 6) having the GMD gene fragment NbiMD (X-B) inserted was purified from the bacterial body with the Wizard Plus Minipreps DNA Purification System (Promega), sequence analysis of the inserted gene fragment was carried out to verify that the GMD gene fragments were flanking the intron sequence and formed an inverted repeat sequence.

(5) Transformation of Tobacco Nicotiana benthamiana

The transformation of tobacco (N. benthamiana) was carried out using Agrobacterium tumefaciens LBA4404 strain, by way of the leaf disc method. From a tobacco leaf treated by sterilization, a leaf disc approximately 1 cm in diameter was cut out with a cork borer, soaked in a bacterial solution of Agrobacterium tumefaciens LBA4404 strain having GMDinv-pBE2113, and co-cultured (23° C., 16 hours illumination) on MS agar medium for 2 days to let Agrobacterium infect the tobacco leaf.

On the third day, Agrobacterium bacterial body was eliminated by washing the leaf disc with an MS liquid medium containing 5 mg/l kanamycin and 500 mg/l carbenicillin. The leaf disc was cultured over a redifferentiation MS agar medium added with these antibiotics to obtain a transformed tobacco shoot. The shoot was cultured in a rooting medium containing kanamycin and carbenicillin, and after sufficient rooting, was grown in a greenhouse.

(6) Evaluation by MALDI-TOF-MS of the Effect of the Inhibition on Plant-Type Sugar Chain Modification

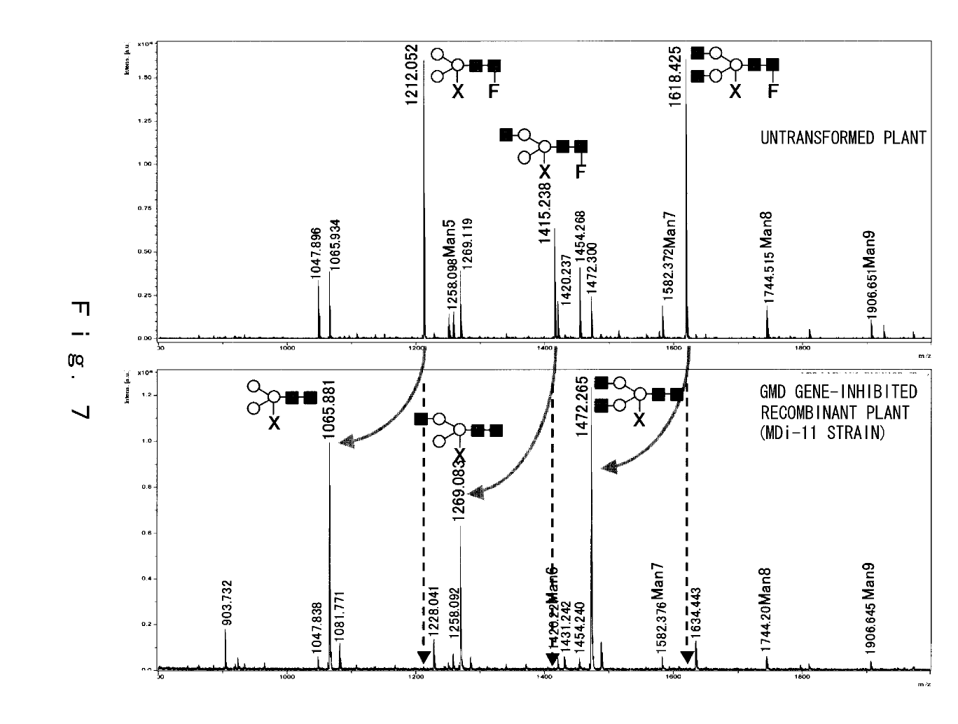

N-linked sugar chain was purified from the obtained transformed plant, and a sugar chain structure analysis by MALDI-TOF-MS was carried out to evaluate the extent of fucose modification inhibition. As a result, it was observed that α-1,3-fucose modification to N-linked sugar chain decreased from 70% to 5% in the Mdi-11 strain (FIG. 7 and Table 2).

TABLE 2

| | Abundance ratio of | |
|---|---|---|
| | {illegible} sugar chain (%) | |
| | | Strain inoculated |
| | | with GMD gene |
| Proposed structure of sugar chain | WT | inhibition vector |
| Man3XylGlcNAc | 0.25 | 0.00 |
| Man2XylGlcNAc2 | 0.00 | 3.32 |
| Man3GlcNAc2 | 0.24 | 0.00 |
| Man2XylGucGlcNAc2 | 4.11 | 0.85 |
| Man2XylFucGlcNAc2 | 1.08 | 0.00 |
| Man3XylGlcNAc2 | 5.26 | 22.04 |
| Man3FucGlcNAc2 | 0.00 | 1.86 |
| GlcNAcMan2XylGlcNAc2 | 0.41 | 0.00 |
| GlcNAcMan3GlcNAc2 | 0.20 | 0.00 |
| Man3XylFucGlcNAc2 | 21.35 | 0.00 |
| Man4XylGlcNAc2 | 0.42 | 3.09 |
| GlcNAcMan2XylFucGlcNAc2 | 1.96 | 0.45 |
| Man5 | 2.00 | 1.46 |
| GlcNAcMan3XylGlcNAc2 | 5.24 | 16.16 |
| GlcNAcMan3FucGlcNAc2 | 0.00 | 0.68 |
| GlcNAc2Man3GlcNAc2 | 0.34 | 0.38 |
| GlcNAcMan3XylFucGlcNAc2 | 8.77 | 0.00 |
| Man6 | 2.83 | 1.45 |
| HexHexNAcMan3Xyl GlcNAc2 | 0.00 | 1.46 |
| GlcNAc2Man2XylFucGlcNAc2 | 5.82 | 0.67 |
| GlcNAc2Man3XylGlcNAc2 | 3.46 | 37.72 |
| HexGlcNAcMan3XylFucGlcNAc2 | 0.48 | 0.00 |
| Man7 | 2.60 | 1.58 |
| GlcNAc2Man3XylFucGlcNAc2 | 25.91 | 0.00 |
| HexHexNAc2Man3XylGlcNAc2 | 0.17 | 3.57 |
| HexGlcNAc2Man3FucGlcNAc2 | 0.36 | 0.00 |
| Man8 | 2.64 | 1.70 |
| FucHexGlcNAc2Man3FucGlcNAc2 | 0.00 | 0.21 |
| Hex2GlcNAc2Man3FucGlcNAc2 | 0.90 | 0.35 |
| Man9 | 1.70 | 1.01 |
| FucHexGlcNAc2Man3XylFucGlcNAc2 | 1.04 | 0.00 |
| Hex3GlcNAc2Man3FucGlcNAc2 | 0.47 | 0.00 |
| Total | | |
| Fuc modification present | 72.25 | 5.06 |
| No Fuc | 15.99 | 87.74 |
| High-mannose | 11.77 | 7.19 |

(7) Evaluation by RT-PCR of the Expression Pattern of the GMD Gene

A single-strand cDNA was synthesized with the Ready-To-Go RT-PCR Beads (GE Health Care) using 3 μg of total RNA sample and oligo-dT primers. PCR using GoTaq Green Master Mix (Promega) and T-personal Thermal Cycler (Biometra) was carried out with 1 μl of the present cDNA solution as sample. For the PCR, after treatment thereof at 94° C. for 3 minutes, the cycle 94° C. (30 seconds)→56° C. (30 seconds)→72° C. (1 minute 30 seconds) was repeated 35 times.

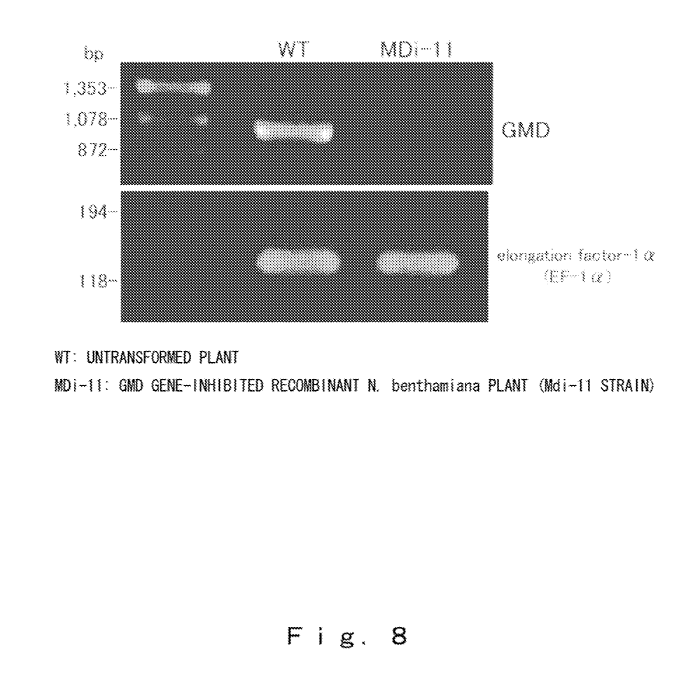

GMD gene primers, NbMD (F) (SEQ ID NO. 1: 5′-AAGTAGCTCTGATCACCGGC) and NbMD (R) (SEQ ID NO. 2: 5′-TAACTCAATATCCTCGTCCACC) were used. In addition, with the mRNA derived from the elongation factor-1α gene as an internal standard, a partial sequence thereof was amplified using the primers EF-1-F (SEQ ID NO. 5: 5′-GATTGGTGGTATTGGAACTGTCC) and EF-1-R (SEQ ID NO. 6: 5′-GAGCTTCGTGGTGCATCTC). As a result, in transformed tobacco, a remarkable decrease in the amount of mRNA derived from the GMD gene was observed compared to untransformed tobacco (FIG. 8).

### INDUSTRIAL APPLICABILITY

As described above, the present invention relates to a method for modifying a sugar chain structure in plant and a plant produced by the method, and, according to the present invention, by reducing or inhibiting fucose modification to glycoprotein N-linked sugar chain in plant, a method for modifying a plant-type sugar chain structure can be provided. A transgenic plant can be prepared and provided, in which fucose modification to glycoprotein N-linked sugar chain is reduced or inhibited. By utilizing the transgenic plant as a host, synthesis of a glycoprotein in which the sugar chain structure of the glycoprotein is modified becomes possible. By utilizing the transgenic plant of the present invention, production of a glycoprotein becomes possible, in which plant-specific fucose modification having the possibility of becoming an allergen when administered in an organism is reduced or inhibited.

According to the present invention, inhibition or removal of fucose modification to sugar chain becomes possible in plant, α-1,3-fucose modification to glycoprotein N-linked sugar chain, which is a plant-specific sugar chain modification that has the possibility of becoming an allergen to an animal, is removable, and application of the present invention to the production of medicinal glycoprotein in plant is possible. The present invention is useful as one that enables, with the transgenic plant, the production and provision of a medicinal glycoprotein in which allergenicity is decreased.

1. A method for modifying a sugar chain structure characterized either by introducing a plant transformation vector having inserted therein a gene fragment inhibiting expression of gene coding for an enzyme involved in synthesis of GDP-fucose, which is a species of sugar nucleotide, into a plant or a plant cell to carry out transformation of the plant, or, infecting a plant or a plant cell with a plant virus vector having inserted therein a gene fragment inhibiting expression of gene coding for an enzyme involved in synthesis of GDP-fucose, thereby inhibiting expression of GDP-fucose synthetase gene to reduce or inhibit fucose modification to a plant-type N-linked sugar chain in the plant,
wherein a DNA sequence of the gene coding for the enzyme, a DNA sequence corresponding to a mutant, an allele, a variant or a homolog of the DNA sequence, or a DNA sequence having at least 50% homology to the gene is used as the gene fragment.

2. The method according to claim 1, wherein the reduction or inhibition of fucose modification to the plant-type N-linked sugar chain is a reduction or an inhibition of fucose modification to a sugar chain, including a glycoprotein sugar chain, a glycolipid sugar chain, an oligosaccharide or a polysaccharide of a plant.

3. The method according to claim 1, wherein the expression of the gene coding for the enzyme involved in the synthesis of GDP-fucose, which is a species of sugar nucleotide, transcription or translation of the gene is blocked by transcriptional gene silencing (TGS), post-transcriptional gene silencing (PTGS) or virus-induced gene silencing (VIGS), thereby blocking intracellular synthesis of GDP-fucose and reducing or inhibiting fucose modification to the sugar chain.

4. The method according to claim 1, wherein the gene coding for the enzyme involved in the synthesis of GDP-fucose is a GDP-D-mannose-4,6-dehydratase (GMD) gene or a GDP-keto-6-deoxymannose-3,5-epimerase/4-reductase (GER) gene.

5. The method according to claim 1, wherein a plant virus vector is used as the vector for inducing the inhibition of the gene.

6. The method according to claim 1, wherein a plant transformation vector is used as the vector for inducing the inhibition of the gene.

7. A transgenic plant, a plant cell or a plant obtained by the method according to claim 1, wherein GDP-fucose synthetase gene expression has been inhibited and fucose modification to a sugar chain has been reduced or inhibited.

8. The transgenic plant, the plant cell or the plant according to claim 7, wherein the transgenic plant, the plant cell or the plant is a plant cell, a plant or a progeny thereof.

9. A glycoprotein, a glycopeptide, a glycolipid or a sugar chain obtained from the plant cell, the plant or the progeny thereof defined in claim 8.

10. A method for synthesizing glycoprotein in which fucose modification to a sugar chain has been reduced or inhibited characterized by using the transgenic plant defined in claim 7 as a host.
